(19) United States
(12) Patent Application Publication
(10) Pub. No.: US 2007/0053843 A1
(43) Pub. Date: Mar. 8, 2007

(54) Inhalable pharmaceutical formulations employing lactose anhydrate and methods of administering the same

(76) Inventors: Michelle L. Dawson, Ware (GB); Trevor C. Roche, Ware (GB); Mark Whitaker, Ware (GB); Owen Chisora Chidavaenzi, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,449

(22) PCT Filed: Oct. 22, 2004
(86) PCT No.: PCT/US04/35129

§ 371 Date: Apr. 20, 2006

Related U.S. Application Data
(60) Provisional application No. 60/515,077, filed on Oct. 28, 2003.

(51) Int. Cl. A61K 31/573; A61K 31/138; A61K 9/14; A61L 9/04

(52) U.S. Cl. 424/46; 514/171; 514/651; 514/53

(57) ABSTRACT

Pharmaceutical formulations suitable for inhalation comprise at least one pharmaceutically active medicament and lactose anhydrate.

### FIELD OF THE INVENTION

The invention generally relates to pharmaceutical formulations suitable for inhalation which employ lactose and methods of administering the same.

### BACKGROUND OF THE INVENTION

Inhalers are well known devices for administering medicinal products to the respiratory tract. They are commonly used for local relief of respiratory diseases, but the pulmonary route also provides a conduit for the potential systemic delivery of a variety of medicinal products such as analgesics and hormones.

The two main types of inhalers are the pressurized metered dose inhaler (MDI) and the dry powder inhaler (DPI). The MDI uses a volatile propellant to produce an aerosol cloud containing the active ingredient for inhalation. DPIs deliver the active ingredient in the form of dry powder particles to the respiratory tract. To facilitate targeting to the lung, the active ingredient used within an inhaler is typically less than 5 μm, and consequently inherently cohesive._Dispersion upon aerosolisation is achieved by a combination of the inhaler dispersion mechanics and the formulation.

Dry powder formulations for inhalation commonly comprise at least one micronised active substance and a biologically inert carrier. The latter is used in dry powders for inhalation as a diluent, to facilitate manufacture, and as an aerosolisation aid. It typically comprises defined proportions of finely divided and coarser particles to optimise and control the manufacture of the drug product and delivery of the active ingredient to the lung. The carrier may include any acceptable pharmacologically inert material or combination of materials. The most commonly used excipient in DPIs is α-lactose monohydrate.

Lactose can exist as either the alpha or beta form of the crystal. Beta lactose is an anhydrate and is non-hygroscopic below 97% relative humidity (RH). Above 97% RH, it absorbs moisture and mutarotates to form the alpha-monohydrate. Alpha monohydrate is non hygroscopic. Angberg et al, Int. J. Pharm. 73, 209-220 (1991) disclose employing microcalorimetry at 25° C. to investigate the incorporation of hydrate water in roller-dried anhydrous lactose that consisted of 31% alpha- and 69% beta-lactose. Differential scanning calorimetry and water vapor uptake measurements were also performed. Additionally, Angberg et al. disclose that the anhydrous alpha-lactose can accommodate a water molecule to become alpha-lactose monohydrate. Beta-lactose can only exist as the anhydrous form, but it can mutarotate to alpha-lactose and subsequently incorporate water.

The performance of dry powder inhalers is typically affected by the environmental conditions in which they are stored and used, unless the formulation is protected in some way from the environment. In particular, high relative humidity of the ambient air is believed to adversely affect the physical stability and the in vitro performance of the powder. For example, Jashnani et al (Int. J. Pharm. 113, 123-130. 1995) disclose a decrease in fine particle dose or fine particle percent for both albuterol and albuterol sulfate with increasing relative humidity at any given temperature with differences being more marked at higher temperatures. Ganderton and Kassem (Advances in Pharm Sci. 165-191, 1992) disclose that high relative humidity results in an increase in adhesive forces between drug and carrier due to capillary action. Hickey et al (Pharm. Tech. 58-82, 1994) disclose that interparticle cohesion usually increases as the relative humidity of the air increases. At humidities greater than 65% fluid condenses in the space between particles that are close together. This can lead to liquid bridges between neighboring particles, and the effect of surface tension gives rise to attractive forces. Additionally, Jashnani et al (Int. J. Pharm, 130, 13-24, 1996) disclose a comparison of aerosols formed by three salts and the free base of albuterol following their formation from similarly micronized crystalline powders held in a model dry powder inhaler under varying environmental conditions. Overall, Jashnani et al disclose that albuterol stearate, the most hydrophobic salt, emptied and aerosolized best from the inhaler and showed least sensitivity to temperature and humidity.

Various methodologies have been employed in an attempt to assess and prevent the drop in physical performance induced by adverse environmental storage. Maggi et al (Int. J. Pharm. 177, 1, 83-91, 1999)) disclose employment of an accelerated stability test on two prototypes of a new dry powder inhaler to verify the influence of moisture uptake on the performance of the device. The reservoir based multi dose dry powder inhalers (e.g., Turbuhaler® made commercially available by Astra Zeneca of Wilmington, Del. (see e.g., Wetterlim (Pharm. Res 5, 506-508, 1988)) contain a desiccant store in such inhalers. Williams et al (STP Pharma Sci 19(3) 243-250, 2000) have demonstrated that the inclusion of moisture scavengers within MDI systems helped minimize the undesired consequences caused by moisture ingress into the MDI canisters.

The use of a desiccant integral to the device has also been shown to enhance chemical stability of inhaled products. For example, Wu et al (WO 2000/078286) disclose a medicinal aerosol steroid solution formulation product with enhanced chemical stability. The steroid is a 20-ketosteroid having an OH group at the C-17 or C-21 position and the aerosol container has a non-metal interior surface which has been found to reduce chemical degradation of such steroids.

Alternatively the susceptibility of physical performance dry powder formulations to environmental humidity may be potentially reduced by increasing the moisture resistance of the dry powder formulation to the environment. Keller and Mueller-Waltz (WO 2000/028979) disclose the use of magnesium stearate for improving the resistance to moisture, i.e., for lowering the sensitivity of powder mixtures to moisture. Such a concept has also been disclosed for formulations containing formoterol fumarate, salbutamol sulphate and salbutamol base by Mueller-Waltz et al (Drug Delivery to the Lungs XI, The Aerosol Society, London, 2000, 26-29).

The use of dehydrated lactose forms have been disclosed. More specifically, Figura and Epple, Journal of Thermal Analysis, 44, (1995) 45-53 disclose an investigation of dehydrated lactose forms α_{H} and α_{S} by time- and temperature-resolved X-ray powder diffraction and differential scanning calorimetry.

For all of the above disclosures to be used in practice, the desiccant or ternary agent should be either non-inhaled, or safety data generated to demonstrate the clinical acceptability of any additional inhaled excipients within the formulation. As such, there exists a desire for excipients for use within inhalation formulations to manifest a physical stability enhancing contribution to the formulation. There is also a need in the art to address potential problems associated with stability problems and a decrease in fine particle mass as a function of storage length, i.e., the time commencing with the point at which the formulation is placed within the inhalation device. As known in the art, “fine particle fraction” or FP Fraction refers to the percentage of particles within a given dose of aerosolized medicament that is of “respirable” size, as compared to the total emitted dose. It is highly desirable to provide a pharmaceutical formulation which produces a consistent FP Fraction throughout the life of the product.

### SUMMARY OF THE INVENTION

In one aspect, the invention provides a pharmaceutical formulation suitable for inhalation comprising at least one pharmaceutically active medicament and lactose anhydrate.

In another aspect, the invention provides a method for treating a respiratory disorder in a mammal. The method comprising administrating a therapeutically effective amount of the pharmaceutical formulation to the mammal.

In another aspect, the invention provides an inhalation device employing a pharmaceutical formulation.

The present invention offers a number of surprising advantages and benefits. For example, the present invention is highly advantageous in that it provides inhalable pharmaceutical formulations which are capable of displaying improved desiccating ability, particularly at lower relative humidity conditions. Moreover, the inhalable pharmaceutical formulations are capable of exhibiting improved FP Fraction stability relative to conventional inhalable formulations. Moreover, it is believed that the chemical degradation of the active material can be mediated by the presence of moisture in such formulations. The inhalable pharmaceutical formulations are thus capable of increased chemical stability of the active material relative to conventional formulations. Surprisingly, the pharmaceutical formulations of the invention are capable of exhibiting little, if any, aggregation upon storage, notwithstanding the moisture absorption capabilities of the formulations.

### BRIEF DESCRIPTION OF THE DRAWINGS

FIG. 1 is a chart illustrating the X-Ray diffraction patterns for anhydrous lactose in comparison with alpha lactose monohydrate.

FIG. 2 is a graph illustrating GVS moisture uptake for various types of lactose.

FIG. 3 is a graph illustrating the weight change of various types of anhydrous lactose upon extended storage at 25° C./75% RH.

FIG. 4 is a graph illustrating FP Fraction values for various formulation blends containing different levels of various types of anhydrous and monohydrate lactose.

FIG. 5 is a graph illustrating the moisture uptake of anhydrous lactose (coarse and fines) and monohydrate lactose.

FIG. 6 is a graph illustrating the moisture uptake of various formulation blends containing different levels of anhydrous (fine and coarse) and monohydrate lactose upon exposure to 25° C./40% RH.

FIG. 7 is a graph illustrating the calculated percent rehydration for various formulation blends containing different levels of anhydrous (fine and coarse) and monohydrate lactose upon storage at 25° C./40% RH.

FIG. 8 is a graph illustrating the equilibrium relative humidity (ERH) of various formulation blends containing different levels of anhydrous (fine and coarse) and monohydrate lactose.

FIG. 9 is a graph illustrating the desiccant capacity of various formulation blends containing different levels of anhydrous (fine and coarse) and monohydrate lactose.

FIG. 10 is a graph illustrating FP Fraction values for various formulation blends containing different levels of anhydrous (fine and coarse) and monohydrate lactose with storage at 25° C./75% RH.

FIG. 11 is a graph illustrating FP Fraction values for various formulation blends containing different levels of anhydrous (fine and coarse) and monohydrate lactose with storage at 40° C./75% RH.

FIG. 12 is a graph illustrating the desiccant capacity of various formulation blends containing different levels of anhydrous (fine and coarse) and monohydrate lactose.

FIG. 13 is a graph illustrating FP Fraction values for various formulation blends containing different levels of anhydrous (fine and coarse) and monohydrate lactose with storage at 25° C./75% RH.

FIG. 14 is a graph illustrating FP Fraction values for various formulation blends containing different levels of anhydrous (fine and coarse) and monohydrate lactose with storage at 40° C./75% RH.

### DETAILED DESCRIPTION OF THE INVENTION

The invention will now be described with respect to the embodiments set forth herein. It should be appreciated that these embodiments are set forth to illustrate the invention, and that the invention is not limited to these embodiments.

All publications, patents, and patent applications cited herein, whether supra or infra, are hereby incorporated herein by reference in their entirety to the same extent as if each individual publication, patent, or patent application was specifically and individually indicated to be incorporated by reference.

It must be noted that, as used in the specification and appended claims, the singular forms “a”, “an” and “the” include plural referents unless the content clearly dictates otherwise.

In one aspect, the invention provides a pharmaceutical formulation suitable for inhalation. The pharmaceutical formulation comprises at least one pharmaceutically active medicament and lactose anhydrate. In one embodiment, the pharmaceutical formulation consists essentially of at least one pharmaceutically active medicament and lactose anhydrate. In one embodiment, the pharmaceutical formulation consists of at least one pharmaceutically active medicament and lactose anhydrate.

Advantageously, the pharmaceutical formulation exhibits a weight gain of at least 0.3 percent when equilibrated at 25° C. and 40 percent RH. More preferably, the formulation exhibits a weight gain of at least 0.2 percent when equilibrated 25° C. and 30 percent RH. Most preferably, the formulation exhibits a weight gain of at least 0.1 percent when equilibrated at 25° C. and 20 percent RH. For the purposes of the invention, the term “equilibrated” is defined as a weight change of less than 0.1% w/w following storage for 4 hours.

For the purposes of the invention, the term “lactose” as used herein is to be broadly construed. As an example, lactose is intended to encompass crystalline, amorphous, isomeric and polymorphic forms of lactose, including, but not limited to, lactose monohydrate, the stereoisomers α-lactose monohydrate and β-anhydrous lactose, as well as alpha-anhydrous lactose. Lactose (i.e., milk sugar) is preferably obtained from cheese whey, which can be manufactured in different forms depending on the process employed. As used herein, the term “particle” is to be broadly interpreted to encompass those of various shapes, sizes, and/or textures which can include those that may have varying degrees of irregularities, disuniformities, etc. or which may possess regular and/or uniform properties.

The term “lactose anhydrate” is defined to encompass lactose having various levels of water content. For example, in one embodiment, the lactose anhydrate includes less than 1 mole of water (e.g., including, without limitation, water) per mole of lactose. In an embodiment, lactose anhydrate may encompass anhydrous lactose. By virtue of employment of the lactose, the pharmaceutical formulation contains varying levels of water. For example in one embodiment, the pharmaceutical formulation is free of water. In another embodiment, the pharmaceutical formulation is substantially free of water. In another embodiment, the pharmaceutical formulation contains less than or equal to about 1, 2, 3, 4, or 5% w/w of water.

In accordance with the invention, the amount of lactose employed in the formulation is believed to assist in achieving the benefits described herein. For example, in one embodiment, the lactose includes at least 1, 3, or 5% w/w lactose anhydrate, more preferably at least 10% w/w lactose anhydrate. In other embodiments, the lactose includes from, at a lower end 1, 2, 3, 5, 10, 20, 30, or 40% w/w to, at a higher end, 5, 10, 20, 30, 40, 50, 60, 70, 80, 90, or 100% w/w lactose anhydrate. In the above embodiments, the balance of the lactose present is monohydrate lactose.

The lactose anhydrate is preferably present as hygroscopic alpha anhydrous lactose or α_{H} anhydrous lactose. For the purposes of the invention “hygroscopic alpha anhydrous lactose” is characterized by having a crystallographic structure, and anomeric ratio consistent with that of the predominantly alpha form of lactose whilst being essentially anhydrous in nature (represented by the lack of water of crystallization). The alpha-anhydrous form is also hygroscopic in nature as demonstrated by the propensity of the material to sorb water (at least 1% w/w) under low environmental relative humidity (RH) conditions (20% RH) at 25° C. The above properties applies to fully dehydrated lactose. Nonetheless, it should be understood that other hygroscopic properties may be displayed by partially dehydrate forms of lactose encompassed by the invention.

The lactose anhydrate may possess various physical properties. As an example, in one embodiment, the lactose anhydrate has a surface area ranging from, at a lower end, about 0.1, 1, 2, 3, or 4 m²/g to, at a higher end, about 6, 7, 8, 9, or 10 m²/g. In one embodiment, the lactose anhydrate has a porosity ranging from, at a lower end, about 0.0001, 0.005, or 0.001 ml/g to, at a higher end, about 0.05 or 0.01 ml/g, measured using BET N₂ adsorption. In one embodiment, the lactose anhydrate has a beta content ranging from, at a lower end, about 0, 5, 10, 15, 20, or 25% w/w to, at a higher end, about 20, 25, 30, 35, or 40% w/w measured using gas chromatography. In one embodiment, the lactose anhydrate possesses a water content ranging from about 0.001 to about 5 percent measured using thermo-gravimetric analysis. In one embodiment, the lactose anhydrate has a dispersive surface energy (γ^{D}ₛ) ranging from about 30 to about 60 mJm⁻² measured using inverse gas chromatography.

In one embodiment, the lactose anhydrate may encompass both coarse and fine fractions. The relative amounts of coarse and fines employed may be varied in accordance with the present invention. In various embodiments, the coarse and fine fractions have preferred size profiles. For example, when employed in a dry powder device (e.g., Diskus®), the coarse fraction preferably has a volume median diameter (D₅₀) ranging from about 60 to about 90 μm, and a volume of sub-14.2 μm particles ranging from about 0 to about 10% v/v. The fine fraction preferably has a volume median diameter (D₅₀) particle size ranging from about 1 to about 30 μm and a volume of sub 14.2 μm particles ranging from about 30 to about 100% v/v, measured using laser diffraction. In general, in one embodiment, the pharmaceutical formulation of the invention, and in particular the lactose employed, is free or substantially free of particle size change as a result of water uptake when exposed a variety of humidity conditions including, without limitation, those set forth herein.

In addition to the above, the lactose anhydrate employed in accordance with the invention may optionally further be present, to a certain level, in amorphous form. In one embodiment, the lactose anhydrate includes at least 1% w/w of amorphous lactose. In one embodiment, the lactose anhydrate includes at least 10% w/w of amorphous lactose. In other embodiments, the anhydrous lactose includes from, at a lower end 0, 1, 5, 10, 20, 30, or 40% w/w to, at a higher end, 5, 10, 20, 30, 40, 50, 60, 70, 80, 90 or 100% w/w amorphous lactose, based on the lactose weight. The balance in the above embodiments is crystalline lactose anhydrate. The above weight percentages are based on the weight of the lactose.

In general, lactose may be formed by various processes known in the art. One example is set forth in Figura, L. O. and Epple M., J, Thermal Anal., (1995) 44-53. In one embodiment, for example, hygroscopic anhydrous lactose (i.e., α_{H} anhydrous lactose) may be manufactured by a rapid thermal dehydration by heating at 120° C. under 20 mbar pressure for 3 hours. Other processes may also be employed.

Medicaments, for the purposes of the invention, include a variety of pharmaceutically active ingredients, such as, for example, those which are useful in inhalation therapy. In general, the term “medicament” is to be broadly construed and include, without limitation, actives, drugs and bioactive agents, as well as biopharmaceuticals. In various embodiments, medicament may be present in micronized form. Appropriate medicaments may thus be selected from, for example, analgesics, (e.g., codeine, dihydromorphine, ergotamine, fentanyl or morphine); anginal preparations, (e.g., diltiazem; antiallergics, e.g., cromoglicate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., beclometasone dipropionate, fluticasone propionate, flunisolide, budesonide, rofleponide, mometasone furoate, ciclesonide, triamcinolone acetonide or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl)ester)); antitussives, (e.g., noscapine; bronchodilators, e.g., albuterol (e.g. as sulphate), salmeterol (e.g. as xinafoate), ephedrine, adrenaline, fenoterol (e.g. as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-(henylethoxy)propyl]sulfonyl]ethyl]-amino]ethyl-2(3H)-benzothiazolone), 3-(4-{[6-({(2R)-2-hydroxy-2-[4-hydroxy-3-(hydroxymethyl)phenyl]ethyl}amino)hexyl]oxy}butyl) benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol; diuretics, (e.g., amiloride; anticholinergics, e.g., ipratropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament. It will be further clear to a person skilled in the art that where appropriate, the medicaments may be used in the form of a pure isomer, for example, R-salbutamol or RR-formoterol.

Particular medicaments for administration using pharmaceutical formulations in accordance with the invention include anti-allergics, bronchodilators, beta agonists (e.g., long-acting beta agonists), and anti-inflammatory steroids of use in the treatment of respiratory conditions as defined herein by inhalation therapy, for example cromoglicate (e.g. as the sodium salt), salbutamol (e.g. as the free base or the sulphate salt), salmeterol (e.g. as the xinafoate salt), bitolterol, formoterol (e.g. as the fumarate salt), terbutaline (e.g. as the sulphate salt), reproterol (e.g. as the hydrochloride salt), a beclometasone ester (e.g. the dipropionate), a fluticasone ester (e.g. the propionate), a mometasone ester (e.g., the furoate), budesonide, dexamethasone, flunisolide, triamcinolone, tripredane, (22R)-6.alpha.,9.alpha.-difluoro-11.beta.,21-dihydroxy-16.alpha.,17.alpha.-propylmethylenedioxy-4-pregnen-3,20-dione. Medicaments useful in erectile dysfunction treatment (e.g., PDE-V inhibitors such as vardenafil hydrochloride, along with alprostadil and sildenafil citrate) may also be employed. It should be understood that the medicaments that may be used in conjunction with the inhaler are not limited to those described herein.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more medicaments. Formulations containing two active ingredients are known for the treatment of respiratory disorders such as asthma, for example, formoterol (e.g. as the fumarate) and budesonide, salmeterol (e.g. as the xinafoate salt) and fluticasone (e.g. as the propionate ester), salbutamol (e.g. as free base or sulphate salt) and beclometasone (as the dipropionate ester) are preferred.

In one embodiment, a particular combination that may be employed is a combination of a beta agonist (e.g., a long-acting beta agonist) and an anti-inflammatory steroid. One embodiment encompasses a combination of fluticasone propionate and salmeterol, or a salt thereof (particularly the xinafoate salt). The ratio of salmeterol to fluticasone propionate in the formulations according to the present invention is preferably within the range 4:1 to 1:20. The two drugs may be administered in various manners, simultaneously, sequentially, or separately, in the same or different ratios. In various embodiments, each metered dose or actuation of the inhaler will typically contain from 25 μg to 100 μg of salmeterol and from 25 μg to 500 μg of fluticasone propionate. The pharmaceutical formulation may be administered as a formulation according to various occurrences per day. In one embodiment, the pharmaceutical formulation is administered twice daily.

The pharmaceutical formulation may include various amounts of the one or more excipient and lactose anhydrate. As an example, in various embodiments, the formulation may include, at a lower end, from 0.05, 0.1, 1, 2 3, 5, 10, 15, 20, 25 or 30 to, at a higher end 5, 6, 7, 8, 9, 10, 15, 20, 25, 30, 35, 40, 45 or 50% w/w of the at least one pharmaceutically active medicament. The remaining portion of the formulation includes lactose anhydrate, as well as optionally other pharmaceutically inert ingredients.

The pharmaceutical formulations may be present in the form of various inhalable formulations. In one embodiment, the pharmaceutical formulation is present in the form of a dry powder formulation, the formulation of such may be carried out according to known techniques. Dry powder formulations for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base which includes lactose and, optionally, at least one additional excipient (e.g., carrier, diluent, etc.). In various embodiments, each capsule or cartridge may generally contain between 20 μg and 10 mg of the at least one medicament. In one embodiment, the formulation may be formed into particles comprising at least one medicament, and excipient material(s), such as by co-precipitation or coating. When employed as a dry powder, packaging of the formulation may be suitable for unit dose or multi-dose delivery. In the case of multi-dose delivery, the formulation can be pre-metered (e.g., as in Diskus®, see GB 2242134/U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g. as in Turbuhaler, see EP 69715, or in the devices described in U.S. Pat. No. 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet hermetically but peelably sealed thereto to define a plurality of containers, each container having therein an inhalable formulation containing the at least one medicament, the lactose, optionally with other excipients. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. The lid sheet may preferably be peeled from the base sheet in a longitudinal direction from a first end of the base sheet.

In one embodiment, the formulations may be employed in or as suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Such formulations may be delivered via a pressurized inhaler, e.g., a Metered Dose Inhaler (MDI). Exemplary MDIs typically include canisters suitable for delivering the pharmaceutical formulations. Canisters generally comprise a container capable of withstanding the vapour pressure of the propellant used such as a plastic or plastic-coated glass bottle or preferably a metal can, for example an aluminum can which may optionally be anodised, lacquer-coated and/or plastic-coated, which container is closed with a metering valve. Aluminum cans which have their inner surfaces coated with a fluorocarbon polymer are particularly preferred. Such polymers can be made of multiples of the following monomeric units: tetrafluoroethylene (PTFE), fluorinated ethylene propylene (FEP), perfluoroalkoxyalkane (PFA), ethylene tetrafluoroethylene (EFTE), vinyldienefluoride (PVDF), and chlorinated ethylene tetrafluoroethylene. Embodiments of coatings used on all or part of the internal surfaces of an MDI are set forth in U.S. Pat. Nos. 6,143,277; 6,511,653; 6,253,762; 6,532,955; and 6,546,928.

MDIs may also include metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK356) and 3M-Neotechnic Ltd, UK (e.g. Spraymiser™). Embodiments of metering valves are set forth in U.S. Pat. Nos. 6,170,717; 6,315,173; and 6,318,603.

In various embodiments, the MDIs may also be used in conjunction with other structures such as, without limitation, overwrap packages for storing and containing the MDIs, including those described in U.S. Pat. No. 6,390,291, as well as dose counter units such as, but not limited to, those described in U.S. Pat. Nos. 6,360,739 and 6,431,168.

In another aspect, the invention relates to a container suitable for use in conjunction with a pharmaceutical formulation. The container comprises at least one pharmaceutically active medicament and lactose anhydrate. The container is structured such that the formulation possesses moisture sorption properties as described herein. The container may be employed in conjunction with the various inhalation devices described, e.g., dry powder inhalers and metered dose inhalers. If used in a dry powder inhaler, the container may be present in various forms such as, without limitation, those described hereinabove such as a capsule, cartridge, reservoir, as well as a container formed from a base sheet and a lid sheet. If used in a metered dose inhaler, the container may be present as described herein, e.g., as a canister.

The pharmaceutical formulation of the invention may be used to treat a number of respiratory conditions. Such respiratory conditions include, without limitation, diseases and disorders associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis). Accordingly, and in view of the above, in another aspect, the invention provides a method for treating a respiratory disorder in a mammal such as a human. The method comprises administrating a pharmaceutically effective amount of a pharmaceutical formulation as defined herein. For the purposes of the invention, the term “pharmaceutically effective amount” is to be broadly interpreted and encompass the prophylaxis and/or treatment of the disorder.

In another aspect, the invention provides a method of treating a respiratory condition. The method comprises administering to a patient by oral or nasal inhalation a pharmaceutically effective amount of a pharmaceutical formulation by using a device as defined herein.

Advantageously, and in accordance with the present invention, the medicament(s) present in the pharmaceutical formulation is believed to exhibit a more stable FP Fraction relative to medicaments present in conventional inhalable formulations. As an example, in one embodiment, the medicament(s) may experience a decrease in FP Fraction of not greater than 10% from initial following 2.5 months storage at 40° C./75% RH, and/or a drop of no more than 15% from initial following 3 months storage at 25° C./75% RH.

Additionally, the pharmaceutical formulation may exhibit increased chemical stability relative to a similar formulation employing lactose monohydrate. As an example, in one embodiment, the medicament(s) experiences at least 25 percent less degradation as measured by impurity content.

The invention will now be described with respect to the following examples. It should be appreciated that the examples are set forth for illustrative purposes only, and do not limit the scope of the invention as defined by the claims. In the examples, “AF” refers to anhydrous fines and “AC” refers to “anhydrous coarse” as defined above herein. All entries contained various percentages of lactose monohydrate to produce matched concentrations of coarse and fine lactose across the formulations. The Fine Particle Fraction described within the following examples is defined as the amount of active ingredient as a proportion of the total emitted dose, depositing in Stage 2 of a Twin Impinger or Stages 1 to 5 of an Andersen Cascade Impactor, both impactors operating at a vacuum flow rate of 60 Imin⁻¹.

### EXAMPLE 1

### Use of Anhydrous Lactose within Dry Powder Formulations

The effect of various types of anhydrous lactose on FP Fraction stability of dry powder inhalers is illustrated herein.

Two batches of anhydrous lactose were manufactured by thermally dehydrating a coarse classification of lactose monohydrate (MPS 92 μm) under vacuum. This method of dehydration was carried out according to the teachings of Figura, L. O. and Epple M., J, Thermal Anal., (1995) 44-53 purported to produce a stable and a hygroscopic form of anhydrous lactose (as defined by the authors). For the purposes of this example, the manufacturing conditions of the stable and hygroscopic anhydrous lactose are defined as follows:

Stable: 120° C., 985 mbar, 5.5 hr

Hygroscopic 120° C., 20 mbar, 3.5 hr

A third batch of an hydrous lactose was sourced commercially (Anhydrous Lactose NF DT; Quest International, Illinois, US).

### EXAMPLE 2

### Physical Properties of Anhydrous Lactose

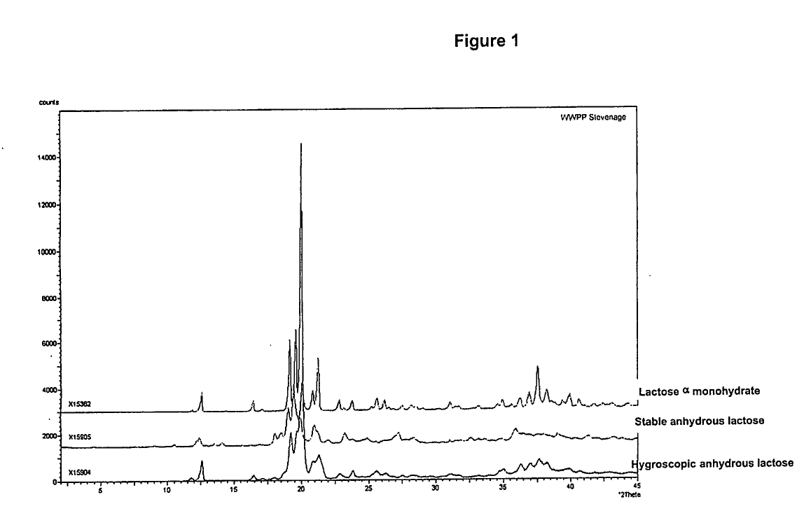

The physical properties of the three anhydrous lactose batches are detailed in Table 1. Included are physical properties of the monohydrate batch used as the input material to produce the two dehydrated lactose batches. FIG. 1 provides a chart illustrating the X-Ray diffraction patterns for the anhydrous lactose in comparison with the lactose monohydrate. The anhydrous nature of the dehydrated forms of lactose is exemplified by the low water contents, whilst the predominance of alpha lactose within the material is demonstrated by the anomeric purity i.e. low beta lactose content. In contrast, whilst the commercial lactose is anhydrous in nature, it contains a high level of beta lactose.

TABLE 1
Physical properties of anhydrous lactose

| | | | | Particle size^{d} | |
|---|---|---|---|---|---|
| | SSA | H₂O content | β content | D₅₀ | |
| | (m²/g)^{a} | (%)^{b} | (%)^{c} | (μm) | % < 14.2 μm |
| Monohydrate | 0.35 | 4.85 | 2.2 | 72.0 | 5.7 |
| Commercial | 0.51 | 0.59 | 75.8 | 59.1 | 14.6 |
| Hygroscopic | 1.5 | 2.03* | 7.5 | 72.5 | 5.3 |
| Stable | 0.53 | N/d | 17.6 | 71.9 | 4.5 |

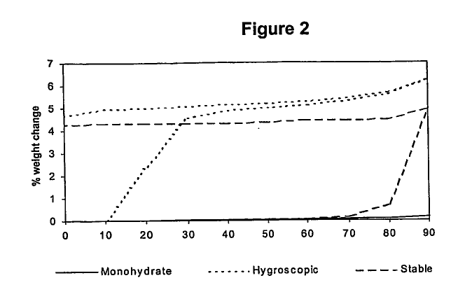

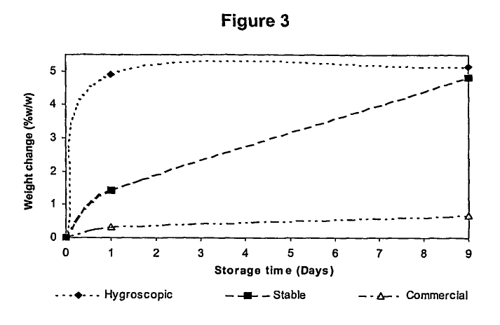

^{a}measured using BET N₂ absorption
^{b}measured using thermo-gravimetric analysis
^{c}measured using gas chromatography
^{d}measured using laser diffraction
n/d not determined
*This value is unduly high and is believed to be due to moisture uptake prior to analysis as it is not consistent with further moisture uptake data detailed in FIGS. 2 and 3

### EXAMPLE 3

### Moisture Uptake of Anhydrous Lactose Batches

The moisture uptake of the lactose batches was measured. FIG. 2 shows the moisture uptake of the two manufactured anhydrous lactose batches, in comparison with the monohydrate control, measured using gravimetric vapor sorption (GVS), and demonstrates different degrees of hygroscopicity between the material. The hygroscopic anhydrous lactose manifests a weight change at significantly lower relative humidity (RH) than the stable anhydrous lactose, although both materials undergo a weight change of approximately 5% w/w, consistent with rehydration.

FIG. 3 illustrates the weight change of the three batches of anhydrous lactose over several days storage at 25° C./75% RH, and demonstrates the differences in hygroscopicity of the materials. This was measured by storing samples of each material at this condition and measuring the weight change from initial at regular timepoints. The hygroscopic alpha anhydrous lactose increases in weight by about 5% within 24 hours, whilst the stable alpha anhydrous lactose achieved this weight gain after nine days. However, the commercial anhydrous lactose only underwent a weight change of less than 1% after nine days storage.

This illustrates the differences in hygroscopicity between the different batches of anhydrous lactose in terms of rate of moisture uptake and critical RH for moisture uptake.

### EXAMPLE 4

### Fine Particle Fraction of Pharmaceutical Formulations

Dry powder blends containing 0.58% w/w salmeterol xinafoate and 0.8% w/w fluticasone propionate were manufactured using a combination of anhydrous lactose and lactose monohydrate with the anhydrous lactose component present in the concentrations described in Table 2.

TABLE 2
Lactose components used to investigate the effect of
anhydrous lactose on physical stability of blends

| | |
|---|---|
| | % Monohydrate |
| % Anhydrous | lactose |

| Lactose | lactose | Coarse^{a} | Fine^{b} |
|---|---|---|---|
| Monohydrate control 1 | 0 | 75 | 25 |
| Commercial | 1 | 76.5 | 22.5 |
| | 10 | 70 | 20 |
| | 60 | 36.5 | 3.5 |
| Stable | 1 | 76 | 23 |
| | 10 | 67 | 23 |
| | 60 | 17 | 23 |
| Hygroscopic | 1 | 75 | 24 |
| | 10 | 67 | 23 |
| Monohydrate control 2 | 0 | 75 | 25 |

^{a}Coarse classification of lactose (MPS 92 μm)
^{b}Fine classification of lactose (MPS 23 μm)

The particle size distributions of the blends were matched using lactose monohydrate. Control batches were manufactured using lactose monohydrate. The lactose blends were manufactured in situ using a high shear blender, and sufficient lactose blend removed to enable addition of the active ingredients in order to achieve to desired drug concentrations. The formulation was manufactured according to methodology described in EP416951 and filled into MDPI foil strips (see e.g., U.S. Pat. No. 5,860,419) using perforated bed filling methodology.

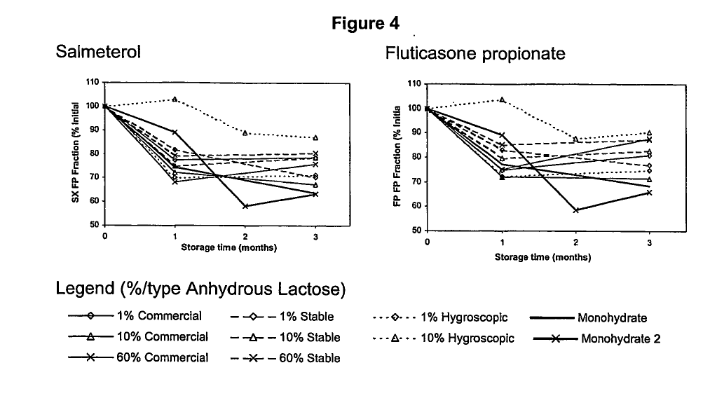

The change in Fine Particle Fraction of the dry powder formulations following storage at elevated temperature and humidity are shown in FIG. 4 and Table 3. These data illustrate the smaller drop in Fine Particle Fraction of both salmeterol and fluticasone propionate of the dry powder formulation containing stable and hygroscopic alpha anhydrous lactose, in comparison with the dry powder formulation containing lactose monohydrate. Dry powder formulations containing hygroscopic alpha anhydrous lactose performed better on stability than those containing stable alpha-anhydrous lactose, demonstrated by the lower drop in Fine Particle Fraction from initial.

TABLE 3
Drop in FP Fraction from initial of anhydrous lactose based
dry powder formulations following 3 months storage at 25° C./75% RH

| Anhydrous % w/w | Salmeterol | Fluticasone Propionate |
|---|---|---|
| 1% Stable | 29.7 | 23.0 |
| 10% Stable | 21.3 | 17.3 |
| 60% Stable | 24.2 | 12.7 |
| 1% Commercial | 21.4 | 19.0 |
| 10% Commercial | 32.7 | 28.7 |
| 60% Commercial | 24.2 | 12.2 |
| 1% Hygroscopic | 28.5 | 25.1 |
| 10% Hygroscopic | 12.7 | 9.5 |
| Monohydrate 1 | 36.3 | 31.7 |
| Monohydrate 2 | 36.7 | 34.0 |

### EXAMPLE 5

### Use of Hygroscopic Anhydrous Lactose within Dry Powder Formulations

Fine and coarse classifications of lactose monohydrate (MPS 23 μm and 92 μm respectively) were thermally dehydrated under vacuum (120° C., 20 mbar) until they had achieved a weight loss of 5% w/w. This dehydration method is purported to produce a hygroscopic form of anhydrous lactose (Figura, L. O. and Epple M., J, Thermal Anal., (1995) 44-53)

Physical Properties of Dehydrated Lactose

Effect of Dehydration on Physical Properties

The physical properties of the following types of lactose are determined and compared as set forth in Table 4.

TABLE 4
Physical properties of dehydrated lactose and monohydrate

| | β content^{a} | SSA^{b} | Porosity^{c} | H₂O content^{d} | DSE^{e} | Particle size^{f} | |
|---|---|---|---|---|---|---|---|
| Lactose type | (%) | (m²/g) | (ml/g) | (%) | mJm⁻² | % <14.2 μm | D50 (μm) |
| Monohydrate | 2.05 | 0.22 | 0.0006 | 5.16 | 33.31 | 5.9 | 71.1 |
| coarse | | | | | | | |
| Anhydrous coarse | 7.25 | 1.53 | 0.0041 | 0.58 | 42.66 | 5.8 | 71.6 |
| Monohydrate | 2.35 | 0.69 | 0.0014 | 5.27 | n/p | 33.6 | 21.8 |
| Fines | | | | | | | |
| Anhydrous fines | 8.75 | 1.88 | 0.0061 | 0.28 | 45.4 | 33.2 | 22.1 |

^{a}measured using gas chromatography
^{b,c}measured using BET N₂ sorption
^{d}measured using thermo-gravimetric analysis
^{e}measured using inverse gas chromatography
^{f}measured using laser diffraction
n/p not performed

As seen, there appear to be little if any significant differences in physical properties with variations in particle size. Dehydration does not appear to affect the particle size of either size classification of lactose. The material is shown to be anhydrous by its low water content.

Moisture Uptake of Dehydrated Lactose

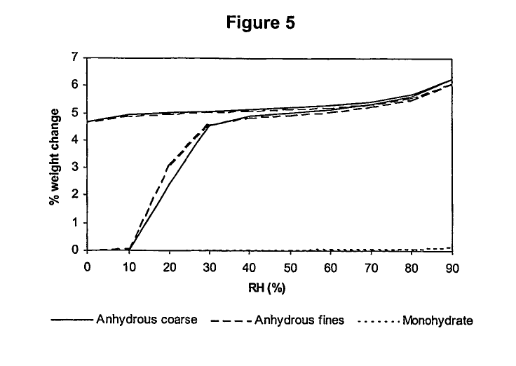

The results are set forth in FIG. 5. As shown, anhydrous lactose is capable of being significantly more hygroscopic than the monohydrate taking up of greater than 5% w/w water at an RH of up to approximately 90 percent. As shown from FIG. 5, the rate and magnitude of water uptake appear to be not significantly dependent on particle size.

### EXAMPLE 6

### Effect of Storage on Physical Properties of Dehydrated Lactose

Samples of the two dehydrated lactose batches were stored at 33 and 58% RH, for about 5 days, until they had undergone a weight increase of 5%, consistent with rehydration. The physical properties of the samples (Table 5) show no change in particle size, beta content or surface area upon rehydration, demonstrated to have occurred as a result of increase in water content. In particular, gross weight change measurements tend to show that dehydrated lactose is capable of taking up approximately 5 percent moisture following 5 days storage at both 33 percent RH and 58 percent RH, with little if any effect on particle size.

TABLE 5
Physical properties of dehydrated lactose on storage

| | | β content^{a} | SSA^{b} | Porosity^{c} | H₂O content^{d} | DSE^{e} | Particle size^{f} | |
|---|---|---|---|---|---|---|---|---|
| Lactose type | | (%) | (m2/g) | (ml/g) | (%) | mJm⁻² | % <14.2 μm | D50 (μm) |
| Coarse | Initial | 7.25 | 1.53 | 0.0041 | 0.58 | 42.66 | 5.8 | 71.6 |
| | 58% | 6.35 | 1.50 | 0.0073 | 4.86 | 46.03 | 6.0 | 72.7 |
| | 33% | 7.2 | Not performed | | | | | |
| Fines | Initial | 8.75 | 1.88 | 0.0061 | 0.29 | 33.2 | 33.2 | 22.1 |
| | 58% | 7.8 | 1.73 | 0.0090 | 4.77 | 48.5 | 33.4 | 22.2 |

| | | |
|---|---|---|
| 33% | 8.7 | Not performed |

^{a}measured using gas chromatography
^{b,c}measured using BET N2 sorption
^{d}measured using thermo-gravimetric analysis
^{e}measured using inverse gas chromatography
^{f}measured using laser diffraction

### EXAMPLE 7

### Use of Dehydrated Lactose in Dry Powder Formulations

Manufacture of Dry Powder Formulations

The dehydrated coarse and fine lactose batches were used to make dry powder blends containing 0.58% w/w salmeterol xinafoate and 0.8% fluticasone propionate according to an experimental design devised to investigate the effect of anhydrous lactose concentration and particle size on Fine Particle Fraction stability. The particle size distributions of the blends were matched using lactose monohydrate (Table 6). The lactose blends were manufactured in situ using a high shear blender, and sufficient lactose blend removed to enable addition of the active ingredients in order to achieve to desired drug concentrations. The formulation was manufactured according to methodology described in EP416951 and filled into MDPI foil strips (see e.g., U.S. Pat. No. 5,860,419) using perforated bed filling methodology (WO00/71419).

TABLE 6
Lactose components used for dry powder formulations

| | Target %/% | Anhydrous | Monohydrate | Anhydrous | Monohydrate |
|---|---|---|---|---|---|
| Batch | AF/AC* | fines % w/w | fines % w/w | coarse % w/w | coarse % w/w |
| 0AF/0AC | 0/0 | 0 | 22 | | 78 |
| 0AF/30AC | 0/30 | 0 | 22 | 30 | 48 |
| 0AF/60AC | 0/60 | 0 | 22 | 60 | 18 |
| 11AF/0AC | 11/0 | 11 | 11 | 0 | 78 |
| 11AF/30AC | 11/30 | 11 | 11 | 30 | 48 |
| 11AF/60AC | 11/60 | 11 | 11 | 60 | 18 |
| 22AF/0AC | 22/0 | 22 | 0 | 0 | 78 |
| 22AF/30AC | 22/30 | 22 | 0 | 30 | 48 |
| 22AF/60AC | 22/60 | 22 | 0 | 60 | 18 |
| 22AF/78AC | 22/78 | 22 | 0 | 78 | 0 |

*AF/AC Anhydrous fines/anhydrous coarse

Water Uptake of Dry Powder Formulations

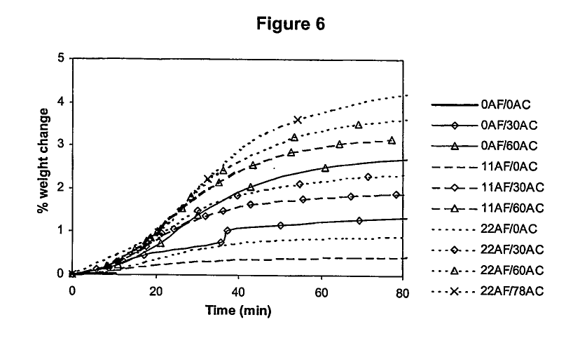

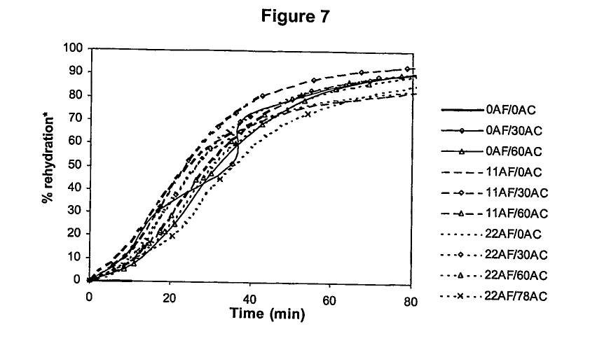

The weight change of the powder formulations was measured under storage at 25° C./40% RH using gravimetric vapor sorption. FIG. 6 shows that the weight change upon storage increases with the concentration of anhydrous lactose within the formulation. When the weight change is translated into the degree of rehydration of the anhydrous lactose component within each formulation (FIG. 7), the rate and degree of rehydration of each formulation is similar, regardless of anhydrous lactose content or particle size.

Particle Size of Pharmaceutical Formulations Following Storage

Samples of the formulations described in Table 6 containing 0.58% salmeterol xinafoate and 0.8% w/w fluticasone propionate were stored at ambient temperature/58% RH for 7 days. The particle size of the formulations, defined here as the volume percentage of particles less the 14.2 μm measured using laser diffraction, are shown in Table 7. The formulations using anhydrous lactose undergo a similar small reduction in fines following storage following storage at 58% RH, to a control lactose monohydrate formulation.

TABLE 7
Particle size of dry powder formulations following storage

% less than 14.2 μm

| Lactose AF/AC % | Initial | Post-storage |
|---|---|---|
| 0/0 | 16.6 (0.27) | 14.4 (0.46) |
| 0/30 | 17.0 (0.46) | 13.7 (0.07) |
| 0/60 | 13.7 (0.09) | 11.1 (0.14) |
| 11/0 | 17.2 (0.41) | 15.1 (0.67) |
| 11/30 | 14.6 (0.10) | 11.2 (0.08) |
| 11/60 | 16.8 (0.33) | 13.9 (0.16) |
| 22/0 | 17.2 (0.20) | 13.8 (0.16) |
| 22/30 | 17.0 (0.33) | 14.4 (0.46) |
| 22/60 | 16.2 (0.18) | 14.5 (0.12) |
| 22/78 | 16.6 (0.43) | 14.8 (0.40) |

Data presented as mean (SD),
n = 3

Equilibrium Relative Humidity (ERH) of Pharmaceutical Formulations

The Equilibrium Relative Humidity (ERH) was measured during the manufacturing process in order to determine the relative humidity within the powder. This parameter represents the relative humidity within the interparticulate void spaces and as such, gives an indication of the ability of the powder to absorb moisture from the immediate storage environment to the extent that it reduces the relative humidity of the bulk powder.

The ERH data or the pharmaceutical formulations described in Table 6 were determined as a function of the filling process. The formulations each contain 0.58% salmeterol xinafoate and 0.8% fluticasone propionate. The ERH was measured by inserting an RH probe into the powder blend on the filling apparatus. This was performed at the start of the filling process, after the manufacture of a sub-batch of MDPI strips (batch 1). Each blend was then left on the filling apparatus for approximately one hour before the manufacture of a second sub-batch of MDPI strips (batch 2). The ERH of the blend was measured at the start and end of the manufacture of this batch.

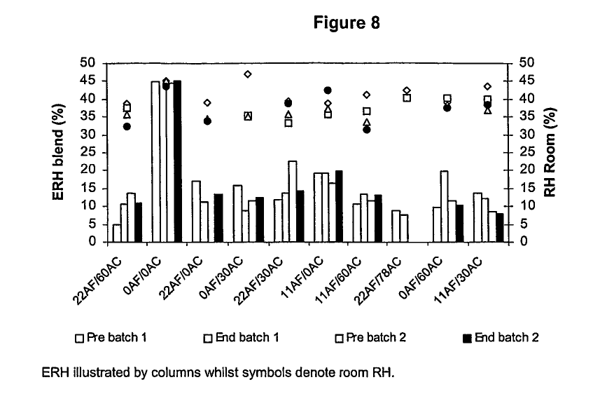

The blends containing various levels of fine and coarse material have a lower ERH relative to blends not containing fine and coarse alpha anhydrous lactose, which is advantageous (FIG. 8). This demonstrates that the dry powder formulations have reduced the water content within the powder bulk, in comparison with the monohydrate control, the ERH of which tracks the relative humidity of the room.

Desiccant Capacity of Pharmaceutical Formulations

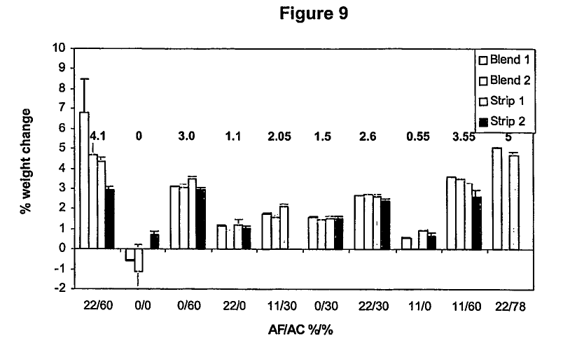

Desiccant capacities of pharmaceutical formulations (0.8% fluticasone propionate and 0.58% salmeterol xinafoate) are determined for various levels of fine and coarse alpha anhydrous lactose, as well as for those employing conventional lactose, i.e., 0/0 AF/AC percent. Desiccant capacity was assessed as the propensity of samples of each formulation to undergo a further water induced weight change upon storage at 58% RH, and is used as an indication of the ability of a formulation to retain a degree of dehydration during a manufacturing process. Naked blends and those blends present in blister strips are evaluated. Samples of blend were taken at the start of the filling process and having been exposed to the environment on the filling apparatus for approximately one hour (labeled 1 and 2 respectively). Blend was tested from two batches of MDPI strip—one manufactured upon immediate exposure of blend, and one after the blend had been exposed to the environment for approximately one hour. The strips were tested approximately 4 weeks after filling, having been stored under ambient environment conditions. FIG. 9 illustrates the results. The text represents the expected percentage weight change, had no rehydration occurred during the filling process. These data suggest that the dry powder formulations containing anhydrous lactose appear to not significantly rehydrate during the manufacturing process, such that they retained their desiccant capacity within the MDPI strip up to four weeks post filling.

As shown, the blends and strips having the fine and coarse fractions generally demonstrate greater desiccating ability relative to those utilizing conventional monohydrate lactose.

Fine Particle Fraction of Pharmaceutical Formulations

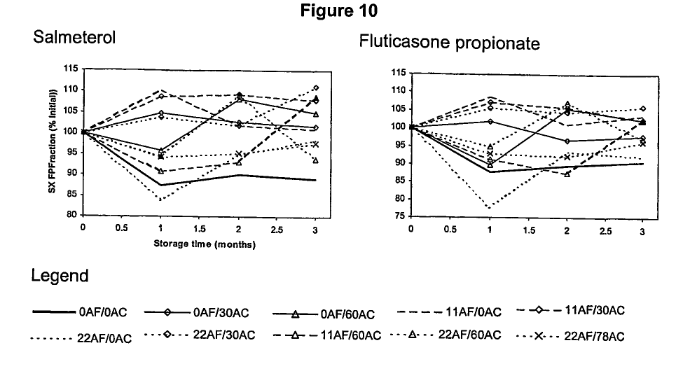

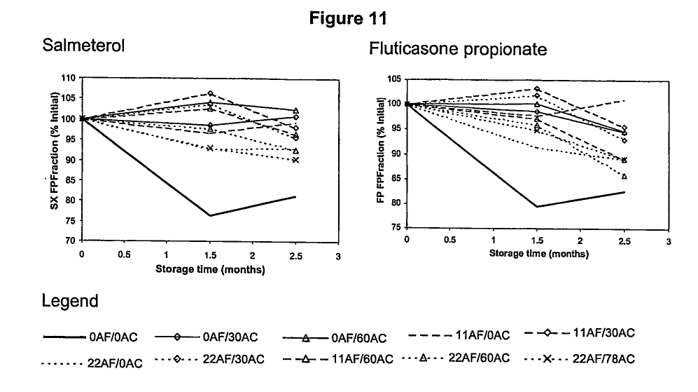

The FP Fraction for salmeterol and fluticasone propionate of formulations following storage at 25° C./75% RH and 40° C./75% RH are determined for dry powder formulations containing various levels of fine and coarse alpha anhydrous lactose, as well as for those employing conventional lactose, i.e., 0/0 AF/AC percent. The formulations are employed in strips for use in a dry powder Diskus® inhaler. FIGS. 10 and 11 illustrate the results.

The drop in Fine Particle Fraction from initial following storage at 25° C./75% RH and 40° C./75% RH are tabulated in Tables 8 and 9. The dry powder formulations containing hygroscopic anhydrous lactose generally exhibit a lower drop in Fine Particle Fraction of both salmeterol and fluticasone on storage in comparison with the lactose monohydrate formulation.

TABLE 8
Drop in Fine Particle Fraction of dry powder formulations
containing anhydrous lactose following
3 months storage at 25° C./75% RH

Drop in Fine particle
Fraction from Initial (%)

| Lactose type (AF/AC %) | Salmeterol | Fluticasone propionate |
|---|---|---|
| 0AF/0AC | 10.8 | 9.4 |
| 0AF/30AC | −1.6 | 2.3 |
| 0AF/60AC | −4.9 | −2.2 |
| 11AF/0AC | −1.2 | −3.7 |
| 11AF/30AC | −8.0 | −2.4 |
| 11AF/60AC | −8.8 | −2.6 |
| 22AF/0AC | 1.5 | 7.7 |
| 22AF/30AC | −11.1 | −6.0 |
| 22AF/60AC | 6.2 | 2.8 |
| 22AF/78AC | 2.3 | 3.8 |

TABLE 9
Drop in Fine Particle Fraction of dry powder formulations
containing anhydrous lactose following 2.5 months storage at
40° C./75% RH

| | |
|---|---|
| | Drop in Fine particle |
| Lactose type | Fraction from Initial (%) |

| (AF/AC %) | Salmeterol | Fluticasone propionate |
|---|---|---|
| 0AF/0AC | 18.8 | 17.4 |
| 0AF/30AC | −0.7 | 5.3 |
| 0AF/60AC | −2.4 | 5.2 |
| 11AF/0AC | 0.6 | −1.2 |
| 11AF/30AC | 2.0 | 4.3 |
| 11AF/60AC | 3.5 | 10.8 |
| 22AF/0AC | 7.0 | 10.8 |
| 22AF/30AC | 4.3 | 7.7 |
| 22AF/60AC | 7.4 | 14.1 |
| 22AF/78AC | 9.7 | 10.9 |

Chemical Stability of Dry Powder Formulations

The chemical stability of formulations following storage at 40° C./75% RH is determined for dry powder formulations containing various levels of fine and coarse alpha anhydrous lactose, as well as for those employing conventional lactose, i.e., 0/0 AF/AC percent. This was assessed by performing a drug related impurity analysis on dry powder blend emptied from MDPI strips that had been on stability for 2.5 months. The resultant chromatograms of the assay were compared and the level of 1-Hydroxy-4-(2-hydroxy-5-{1-hydroxy-2-[6-(4-phenyl-butoxy)-hexylamino]-ethyl}benzyl)-naphthalene-2-carboxylic acid, the principal degradation product within each formulation, quantified. Results are detailed in Table 10.

TABLE 10
1-Hydroxy-4-(2-hydroxy-5-{1-hydroxy-2-[6-(4-phenyl-
butoxy)-hexylamino]-ethyl}-benzyl)-naphthalene-2-carboxylic acid
content of dry powder formulations containing anhydrous lactose
following 2.5 months storage at 40° C./75% RH

| | 1-Hydroxy-4-(2-hydroxy-5-{1- |
|---|---|
| | hydroxy-2-[6-(4-phenyl- |
| | butoxy)-hexylamino]-ethyl}- |
| | benzyl)-naphthalene-2- |
| Anhydrous lactose (%/% AF/AC) | carboxylic acid (% w/w) |
| 0/0 | 1.65 |
| 22/0 | 0.85 |
| 11/30 | 0.41 |
| 0/60 | 0.39 |
| 22/60 | 0.52 |

The concentration of 1-Hydroxy-4-(2-hydroxy-5-{1-hydroxy-2-[6-(4-phenyl-butoxy)-hexylamino]-ethyl}-benzyl)-naphthalene-2-carboxylic acid is highest in the dry powder formulation containing conventional lactose monohydrate i.e. 0/0 AF/AC percent. The chromatographic data show that the dry powder formulations employing anhydrous lactose contain lower levels of drug related impurities, particularly 1-Hydroxy-4-(2-hydroxy-5-{1-hydroxy-2-[6-(4-phenyl-butoxy)-hexylamino]-ethyl}benzyl)-naphthalene-2-carboxylic acid, than the monohydrate based dry powder formulation.

### EXAMPLE 9

### Use of Hygroscopic Anhydrous Lactose within Dry Powder Formulations

The dehydrated coarse and fine lactose batches described in Example 5 were used to make dry powder blends containing 0.58% w/w salmeterol xinafoate and 0.4% fluticasone propionate with varying concentration of anhydrous fine and coarse lactose, as described in Table 11. The particle size distributions of the blends were matched using lactose monohydrate. The lactose blends were manufactured in situ using a high shear blender, and sufficient lactose blend removed to enable addition of the active ingredients in order to achieve to desired drug concentrations. The formulation was manufactured according to methodology described in EP416951 and filled into MDPI foil strips (see e.g., U.S. Pat. No. 5,860,419) using perforated bed filling methodology (WO00/71419).

TABLE 11
Lactose components used to make dry powder formulations

| Lactose | Anhydrous | Monohydrate | Anhydrous | Monohydrate |
|---|---|---|---|---|
| AF/AC % | fines % | fines % | coarse % | coarse % |
| 0AF/0AC | 0 | 22 | 0 | 78 |
| 22AF/60AC | 22 | 0 | 60 | 18 |
| 22AF/78AC | 22 | 0 | 78 | 0 |

### EXAMPLE 10

### Desiccant Capacity of Pharmaceutical Formulations

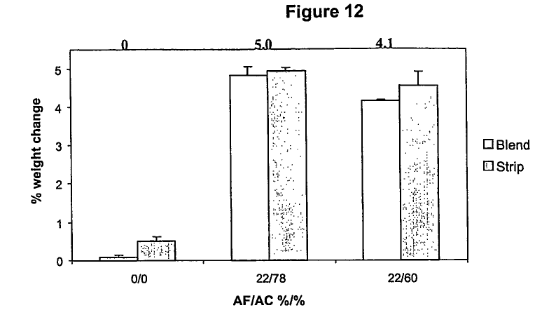

Desiccant capacities of pharmaceutical formulations (0.4% w/w fluticasone propionate and 0.58% w/w salmeterol xinafoate) are determined for various levels of fine and coarse alpha anhydrous lactose, as well as for those employing conventional lactose, i.e., 0/0 AF/AC percent. Desiccant capacity was assessed as the propensity of samples of each formulation to undergo a further water induced weight change upon storage at 58% RH, and is used as an indication of the ability of a formulation to retain a degree of dehydration during a manufacturing process. Naked blends and those blends present in blister strips are evaluated. The strips were tested approximately 4 weeks after filling, having been stored under ambient environment conditions. FIG. 12 illustrates the results. The text represents the expected percentage weight change, had no rehydration occurred during the filling process. These data illustrate that the dry powder formulations containing anhydrous lactose are not believed to significantly rehydrate during the manufacturing process, such that they retained their desiccant capacity within the MDPI strip up to four weeks post filling.

As shown, the blends and strips having the fine and coarse fractions demonstrate greater desiccating ability relative to those utilizing conventional monohydrate lactose.

Fine Particle Fraction of Pharmaceutical Formulations

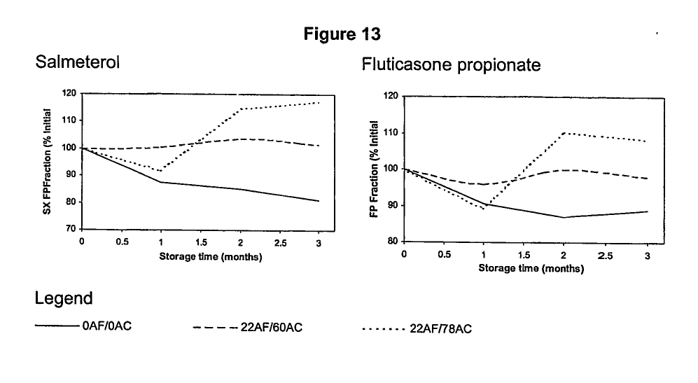

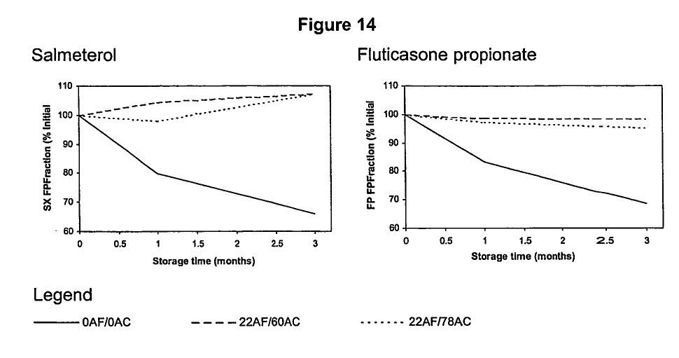

The FP Fraction for salmeterol and fluticasone propionate following storage at 25° C./75% RH and 40° C./75% RH are determined for dry powder formulations, as well as for those employing conventional lactose, i.e., 0/0 AF/AC percent. The formulations are employed in strips for use in a dry powder Diskus® inhaler. FIGS. 13 and 14 illustrate the results. The drop in Fine Particle Fraction from initial following storage at 25° C./75% RH and 40° C./75% RH are tabulated in Tables 12 and 13. The dry powder formulations containing hygroscopic anhydrous lactose exhibit a lower drop in Fine Particle Fraction of both salmeterol and fluticasone on storage in comparison with the lactose monohydrate formulation.

TABLE 12
Drop in Fine Particle Fraction of dry powder formulations
following 3 months storage at 25° C./75% RH.

| | |
|---|---|
| | Drop in Fine particle |
| Lactose % | Fraction from Initial (%) |

| AF/AC | Salmeterol | Fluticasone propionate |
|---|---|---|
| 0AF/0AC | 19.1 | 12.2 |
| 22AF/60AC | −1.7 | 2.0 |
| 22AF/78AC | −17.5 | −8.2 |

TABLE 13
Drop in Fine Particle Fraction of dry powder formulations
following 3 months storage at 40° C./75% RH.

| | |
|---|---|
| | Drop in Fine particle |
| Lactose % | Fraction from Initial (%) |

| AF/AC | Salmeterol | Fluticasone propionate |
|---|---|---|
| 0AF/0AC | 33.9 | 31.4 |
| 22AF/60AC | −2.8 | 1.4 |
| 22AF/78AC | −12.4 | 4.6 |

The invention has been described in reference to the embodiments set forth above. It should be appreciated that such embodiments are for illustrative purposes only, and do not limit the scope of the invention as defined by the claims.

What is claimed is:

1. A pharmaceutical formulation suitable for inhalation, said formulation comprising at least one pharmaceutically active medicament and lactose anhydrate.

2. The pharmaceutical formulation according to claim 1, wherein said formulation exhibits a weight gain of at least 0.3 percent equilibrated 25° C. and 40 percent RH.

3. The pharmaceutical formulation according to claim 1, wherein said formulation comprises at least about 1% w/w of said lactose anhydrate.

4. The pharmaceutical formulation according to claim 1, wherein said formulation is a dry powder formulation.

5. The pharmaceutical formulation according to claim 1, wherein said formulation is an aerosol formulation.

6. The pharmaceutical formulation according to claim 1, wherein said at least one medicament is selected from the group consisting of analgesics, anginal preparations, antiinfectives, antihistamines, anti-inflammatories, antitussives, bronchodilators, diuretics, anticholinergics, hormones, xanthines, therapeutic proteins and peptides, salts thereof, esters thereof, solvates thereof, and combinations thereof.

7. The pharmaceutical formulation according to claim 1, wherein the at least one medicament comprises at least one beta agonist.

8. The pharmaceutical formulation according to claim 7, wherein the at least one beta agonist is selected from the group consisting of salbutamol, terbutaline, salmeterol, bitolterol, formoterol, esters thereof, solvates thereof, salts thereof, and combinations thereof.

9. The pharmaceutical formulation according to claim 7, wherein the at least one beta agonist comprises salmeterol xinafoate.

10. The pharmaceutical formulation according to claim 7, wherein the at least one beta agonist comprises salbutamol sulphate.

11. The pharmaceutical formulation according to claim 1, wherein the at least one medicament comprises at least one anti-inflammatory steroid.

12. The pharmaceutical formulation according to claim 11, wherein the at least one anti-inflammatory steroid is selected from the group consisting of mometasone, beclomethasone, budesonide, fluticasone, dexamethasone, flunisolide, triamcinolone, esters thereof, solvates thereof, salts thereof, and combinations thereof.

13. The pharmaceutical formulation according to claim 11, wherein the at least one anti-inflammatory steroid comprises fluticasone propionate.

14. The pharmaceutical formulation according to claim 1, wherein the at least one medicament comprises at least one beta agonist and at least one anti-inflammatory steroid.

15. The pharmaceutical formulation according to claim 14, wherein the at least one beta agonist comprises salmeterol xinafoate and the at least one anti-inflammatory steroid comprises fluticasone propionate.

16. The pharmaceutical formulation according to claim 1, wherein the at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipatropium, phenylephrine, phenylpropanolamine, pirbuterol, reproterol, rimiterol, isoetharine, tulobuterol, (−)-4-amino-3,5-dichloro-α-[[[6-[2-(2-pyridinyl)ethoxy]hexyl]methyl]benzenemethanol, esters thereof, solvates thereof, salts thereof, and combinations thereof.

17. The pharmaceutical formulation according to claim 1, wherein the at least one medicament is selected from the group consisting of albuterol sulphate, salmeterol xinafoate, fluticasone propionate, beclomethasone dipropionate, and combinations thereof.

18. The pharmaceutical formulation according to claim 1, further comprising at least one additional excipient.

19. The pharmaceutical formulation according to claim 1, wherein the lactose anhydrate comprises amorphous lactose.

20. A pharmaceutical formulation consisting essentially of at least one pharmaceutically active medicament and lactose anhydrate.

21. The pharmaceutical formulation according to claim 20, wherein said formulation exhibits a weight gain of at least 0.3 percent equilibrated 25° C. and 40 percent RH.

22. A method for treating a respiratory disorder in a mammal comprising administrating a pharmaceutically effective amount of a pharmaceutical formulation according to claim 1.

23. The method according to claim 22, wherein the respiratory disorder is selected from the group consisting of asthma, chronic obstructive pulmonary disease (COPD), respiratory tract infection, upper respiratory tract disease, and combinations thereof.

24. The method according to claim 22, wherein said formulation is a dry powder formulation.

25. The method according to claim 22, wherein said formulation is present in an aerosol formulation.

26. The method according to claim 22, wherein said at least one medicament is selected from the group consisting of analgesics, anginal preparations, antiinfectives, antihistamines, anti-inflammatories, antitussives, bronchodilators, diuretics, anticholinergics, hormones, xanthines, therapeutic proteins and peptides, salts thereof, esters thereof, solvates thereof, and combinations thereof.

27. The method according to claim 22, wherein the at least one medicament comprises at least one beta agonist.

28. The method according to claim 27, wherein the at least one beta agonist is selected from the group consisting of salbutamol, terbutaline, salmeterol, bitolterol, formoterol, esters thereof, solvates thereof, salts thereof, and combinations thereof.

29. The method according to claim 27, wherein the at least one beta agonist comprises salmeterol xinafoate.

30. The method according to claim 27, wherein the at least one beta agonist comprises salbutamol sulphate.

31. The method according to claim 22, wherein the at least one medicament comprises at least one anti-inflammatory steroid.

32. The method according to claim 31, wherein the at least one anti-inflammatory steroid is selected from the group consisting of mometasone, beclomethasone, budesonide, fluticasone, dexamethasone, flunisolide, triamcinolone, esters thereof, solvates thereof, salts thereof, and combinations thereof.

33. The method according to claim 31, wherein the at least one anti-inflammatory steroid comprises fluticasone propionate.

34. The method according to claim 22, wherein the at least one medicament comprises at least one beta agonist and at least one anti-inflammatory steroid.

35. The method according to claim 34, wherein the at least one beta agonist comprises salmeterol xinafoate and the at least one anti-inflammatory steroid comprises fluticasone propionate.

36. The method according to claim 22, wherein the at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipatropium, phenylephrine, phenylpropanolamine, pirbuterol, reproterol, rimiterol, isoetharine, tulobuterol, (−)-4-amino-3,5-dichloro-α-[[[6-[2-(2-pyridinyl)ethoxy]hexyl]methyl]benzenemethanol, esters thereof, solvates thereof, salts thereof, and combinations thereof.

37. The method according to claim 22, wherein the at least one medicament is selected from the group consisting of albuterol sulphate, salmeterol xinafoate, fluticasone propionate, beclomethasone dipropionate, and combinations thereof.

38. The method according to claim 22, said formulation further comprising at least one additional excipient.

39. The method according to claim 22, wherein the lactose comprise amorphous lactose.

40. An inhalation device comprising a pharmaceutical formulation contained therein, said pharmaceutical formulation comprising at least one pharmaceutically active medicament and lactose anhydrate.

41. The inhalation device according to claim 40, wherein said formulation includes at least 1% w/w lactose anhydrate, and wherein said formulation exhibits a weight gain of at least 0.3 percent when equilibrated at 25° C. and 40 percent RH.

42. The inhalation device according to claim 40, wherein said inhalation device is a dry powder inhaler.

43. The inhalation device according to claim 42, wherein the dry powder inhaler is a Diskus® inhaler.

44. The inhalation device according to claim 40, wherein said inhalation device is a metered dose inhaler.

45. The inhalation device according to claim 40, wherein said at least one medicament is selected from the group consisting of analgesics, anginal preparations, antiinfectives, antihistamines, anti-inflammatories, antitussives, bronchodilators, diuretics, anticholinergics, hormones, xanthines, therapeutic proteins and peptides, salts thereof, esters thereof, solvates thereof, and combinations thereof.

46. The inhalation device according to claim 40, wherein the at least one medicament comprises at least one beta agonist.

47. The inhalation device according to claim 46, wherein the at least one beta agonist is selected from the group consisting of salbutamol, terbutaline, salmeterol, bitolterol, formoterol, esters thereof, solvates thereof, salts thereof, and combinations thereof.

48. The inhalation device according to claim 46, wherein the at least one beta agonist comprises salmeterol xinafoate.

49. The inhalation device according to claim 46, wherein the at least one beta agonist comprises salbutamol sulphate.

50. The inhalation device according to claim 40, wherein the at least one medicament comprises at least one anti-inflammatory steroid.

51. The inhalation device according to claim 50, wherein the at least one anti-inflammatory steroid is selected from the group consisting of mometasone, beclomethasone, budesonide, fluticasone, dexamethasone, flunisolide, triamcinolone, esters thereof, solvates thereof, salts thereof, and combinations thereof.

52. The inhalation device according to claim 50, wherein the at least one anti-inflammatory steroid comprises fluticasone propionate.

53. The inhalation device according to claim 40, wherein the at least one medicament comprises at least one beta agonist and at least one anti-inflammatory steroid.

54. The inhalation device according to claim 53, wherein the at least one beta agonist comprises salmeterol xinafoate and the at least one anti-inflammatory steroid comprises fluticasone propionate.

55. The inhalation device according to claim 40, wherein the at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipatropium, phenylephrine, phenylpropanolamine, pirbuterol, reproterol, rimiterol, isoetharine, tulobuterol, (−)-4-amino-3,5-dichloro-α-[[[6-[2-(2-pyridinyl)ethoxy]hexyl]methyl]benzenemethanol, esters thereof, solvates thereof, salts thereof, and combinations thereof.

56. The inhalation device to claim 40, wherein the at least one medicament is selected from the group consisting of albuterol sulphate, salmeterol xinafoate, fluticasone propionate, beclomethasone dipropionate, and combinations thereof.

57. The inhalation device according to claim 40, wherein said pharmaceutical formulation further comprises at least one additional excipient.
